(19) United States
(12) Patent Application Publication
(10) Pub. No.: US 2026/0138293 A1
(43) Pub. Date: May 21, 2026

(54) ELBOW-WRIST ROBOTIC TOOL

(71) Applicant: The Hospital for Sick Children, Toronto (CA)

(72) Inventors: Dale Podolsky, Toronto (CA); Catie Balasubramanian, Toronto (CA); Ali Nazari, Toronto (CA); Thomas Looi, Toronto (CA); Sunny Zhang, Toronto (CA)

(21) Appl. No.: 19/391,085

(22) Filed: Nov. 17, 2025

Related U.S. Application Data
(60) Provisional application No. 63/720,908, filed on Nov. 15, 2024.

(51) Int. Cl. B25J 17/02; B25J 9/00; B25J 9/10; B25J 9/12

(52) U.S. Cl. CPC B25J 17/0283; B25J 9/0009; B25J 9/104; B25J 9/12

(57) ABSTRACT

A robotic tool may be shown to provide surgeons with advantages appropriate to a task at hand. During Trans-Oral Robotic Surgery (TORS), instruments traverse through the oral aperture. Aspects of the present application relate to a elbow-wrist robotic tool with a wrist joint at a tool tip, the wrist joint having degrees-of-freedom including wrist pitch, wrist yaw and wrist grip. In addition to a wristed tool tip, aspects of the present application relate to a system wherein the tool includes an elbow joint that may be shown to provide enhanced reachability of the tool within confined workspaces.

### CROSS-REFERENCE TO RELATED APPLICATIONS

This application claims benefit of, and priority from, U.S. Provisional Patent Application No. 63/720,908, filed Nov. 15, 2024, the contents of which are hereby incorporated herein by reference.

### TECHNICAL FIELD

The present disclosure relates, generally, to surgical robots and, in particular embodiments, to robotic tools and, in particular embodiments, to an elbow-wrist robotic tool.

### BACKGROUND

A surgical robot may, generally, be classified as a multi-port system or as a single-port system. Multi-port systems have multiple robotic arms and each robotic arm may direct a corresponding tool. The multiple robotic arms are separated from each other. The multiple robotic arms triangulate towards an operative site. Multi-port systems usually use multiple incisions with trocars through body cavity walls at multiple sites, one site for each arm and tool combination, to reach an operative field. Single-port systems have multiple robotic arms that traverse through a single point. Single-port systems require one incision, with one trocar, to enter a body cavity through a body cavity wall. Single-port systems may be understood to represent an evolution towards single incision, minimally invasive surgery. Existing multi-port surgical robotic systems were developed for large body cavity surgery. Several single-port surgical robotic systems have also been developed to minimize the number of incisions required. Conveniently, single-port surgical robotic systems may be shown to maintain dexterity, visualization and precision at the operative site.

There are advantages and disadvantages of multi-port and single-port surgical robotic systems. Multi-port systems require more incisions. However, multi-port systems may be considered to be less complex than single-port systems, in that the separated arms may require fewer degrees-of-freedom (DOF). Furthermore, triangulation of the arms provides certain benefits with respect to tool tip reach and maneuverability. Single-port systems have the advantage of using a single incision. However, single-port systems may be considered to be more complex than multi-port systems requiring more DOF for each tool and have limitations in the reachability and maneuverability of each tool.

Trans-oral robotic surgery (TORS) involves the utilization of surgical robots to perform surgery within the head and neck of a patient and, in particular, within the oral cavity of the patient. TORS is known to use a trans-oral approach through the oral aperture. TORS is an accelerating field, which takes advantage of various features of surgical robotics. Such features include enhanced access, visualization and precision within confined workspaces, such as the oral cavity. Existing multi-port systems and existing single-port systems have been used for TORS. However, none of the existing systems were developed specifically for TORS and, therefore, have design features that are suboptimal for TORS. Current suboptimal design features include, for example, the presence of a remote center of motion (RCM) for each robotic arm. The RCMs are located at trocars and are designed to be stationary during motion of the arm. The RCMs are important in some cases, such as those that involve port sites through body cavity walls, such as the abdomen and pelvis. However, for TORS, RCMs are less important, as instruments traverse through the oral aperture. In addition, currently available robotic systems have instruments that are too large for the oral cavity workspace and have a sub-optimal tool architecture for the oral cavity workspace. This may be shown to result in frequent collisions between the tools and between the tools and the oral cavity. These collisions can cause tissue injury and may also limit the ability of a given tool to be maneuvered freely within the head and neck through the oral aperture. This may result in awkward tool positions and poses and may significantly impact the ability of a given surgical robot to perform a given surgical task. Finally, existing systems have long and rigid instrument arms that lack the articulation to perform complex tasks within confined workspaces, thereby resulting in frequent tool-tool collisions and tool-cavity collisions.

Similarly, a high dexterity robotic surgical system for pediatric surgery has yet to be developed. Current systems have instrument arms that are too large for pediatric workspaces. Accordingly, a robotic system with tools that are more suitable for the pediatric workspace is desired. Preferably, the robotic system has the dexterity suitable for performing complex surgical tasks.

### SUMMARY

Aspects of the present application relate to a robotic tool designed specifically for TORS and pediatric surgery. During TORS, instruments traverse through the oral aperture and operate within a confined workspace. Similarly, in pediatric surgery, instruments traverse through a body cavity wall or through a body orifice and operate within increasingly smaller workspaces. Therefore, a novel robotic tool that has a more optimal size, with the DOF suitable for performing complex tasks with high dexterity and reachability within these confined workspaces. Aspects of the present application relate to a surgical robotic tool with a wrist joint at a tool tip, the wrist joint having at least two degrees-of-freedom (DOF). The wrist joint DOF includes wrist pitch and wrist yaw. An additional third wrist DOF is grip function, which is attained from the yaw DOF motion where distal links move in opposite directions. In addition to a wristed tool tip, aspects of the present application relate to a system wherein the tool includes an elbow joint that may be shown to provide enhanced reachability of the tool within confined workspaces. The elbow joint may be shown to provide enhanced DOF that may be shown to minimize collisions by allowing for more proximal tool shaft positions and orientations while being able to access the surgical site with a wrist joint that can facilitate highly dexterous and complex surgical tasks within confined workspaces.

According to an aspect of the present disclosure, there is provided a robotic tool. The robotic tool includes a driving unit including a plurality of tool actuators, a tool shaft connected to the driving unit, a three-degree-of-freedom (two-DOF) elbow joint arranged to connect the tool shaft to a secondary shaft, the three-DOF elbow joint configured for control by at least one tool actuator among the plurality of tool actuators and a three-DOF wrist joint connected to the secondary shaft, the three-DOF wrist joint configured for control by at least one tool actuator among the plurality of tool actuators.

### BRIEF DESCRIPTION OF THE DRAWINGS

For a more complete understanding of the present embodiments, and the advantages thereof, reference is now made, by way of example, to the following descriptions taken in conjunction with the accompanying drawings, in which:

FIG. 1A illustrates, in a top-left perspective view, a surgical robot, according to aspects of the present disclosure;

FIG. 1B illustrates the surgical robot of FIG. 1A in an elevation view;

FIG. 2A illustrates the surgical robot of FIG. 1A in a front-right perspective view;

FIG. 2B illustrates the surgical robot of FIG. 1A in a further front-right perspective view;

FIG. 3 illustrates a robotic arm with a tool, according to aspects of the present disclosure;

FIG. 4 illustrates a robotic arm, in isolation, to illustrate five setup joints and a tool, in accordance with aspects of the present application;

FIG. 5 illustrates, in an elevation view, a generic one of the tools associated with a generic one of the robotic arms, in accordance with aspects of the present application;

FIG. 6 illustrates, in a perspective view, the tool of FIG. 5;

FIG. 7 illustrates the degrees-of-freedom (DOF) and motion of main platform joints, in accordance with aspects of the present application;

FIG. 8 illustrates the DOF and motion of robotic arm joints, in accordance with aspects of the present application;

FIG. 9 illustrates an elbow joint implemented as a two-DOF joint, in accordance with aspects of the present application;

FIG. 10 illustrates a two-DOF joint with a yaw function and a pitch function, in accordance with aspects of the present application;

FIG. 11 illustrates four links of a wrist joint, in accordance with aspects of the present application;

FIG. 12A illustrates the DOF and joint axis of a wrist joint, in accordance with aspects of the present application;

FIG. 12B illustrates the wrist joint of FIG. 12A in an example pose, in accordance with aspects of the present application;

FIG. 13 illustrates, in a side view, a custom pin for an elbow joint, in accordance with aspects of the present application;

FIG. 14 illustrates cables prior to entering an elbow joint, in accordance with aspects of the present application;

FIG. 15 illustrates a tendency for a roll function to cause cables to tangle at the center of a tool;

FIG. 16 illustrates isolation of a twisting of cables to a proximal region where a tube redirects a path of the cables in a helical shape, in accordance with aspects of the present application;

FIG. 17A illustrates robotic arm positioning for a multi-port configuration, in accordance with aspects of the present application;

FIG. 17B illustrates robotic arm positioning for a multi-port configuration, in accordance with aspects of the present application;

FIG. 18 illustrates robotic arm positioning for a single-port configuration, in accordance with aspects of the present application;

FIG. 19 illustrates an axial view from below illustrating the robotic arms and the tools adjacent to each other, in accordance with aspects of the present application;

FIG. 20A illustrates the elbow-wrist tool, including the tool shaft, the elbow joint, the secondary shaft and the wrist joint, in accordance with aspects of the present application;

FIG. 20B illustrates the manner in which the wrist joint connects to the tool shaft, in accordance with aspects of the present application;

FIG. 20C illustrates the manner in which the wrist joint connects to the secondary shaft, in accordance with aspects of the present application;

FIG. 21A illustrates example circular cable routing for the elbow joint, in accordance with aspects of the present application;

FIG. 21B illustrates example spiral routing for the elbow joint, in accordance with aspects of the present application;

FIG. 22A illustrates, in a right, front isometric view, the example design for the first tool shaft part, in accordance with aspects of the present application;

FIG. 22B illustrates, in a top plan view, an example design for the first tool shaft part, in accordance with aspects of the present application;

FIG. 22C illustrates, in a front view, the example design for the first tool shaft part, in accordance with aspects of the present application;

FIG. 22D illustrates, in a right side view, the example design for the first tool shaft part, in accordance with aspects of the present application;

FIG. 23A illustrates, in a right, front isometric view, the example design for the first elbow joint part, in accordance with aspects of the present application;

FIG. 23B illustrates, in a top plan view, an example design for the first elbow joint part, in accordance with aspects of the present application;

FIG. 23C illustrates, in a front view, the example design for the first elbow joint part, in accordance with aspects of the present application;

FIG. 23D illustrates, in a left side view, the example design for the first elbow joint part, in accordance with aspects of the present application;

FIG. 24A illustrates, in a right, front isometric view, the example design for the proximal end of the secondary shaft, in accordance with aspects of the present application;

FIG. 24B illustrates, in a top plan view, an example design for the proximal end of the secondary shaft, in accordance with aspects of the present application;

FIG. 24C illustrates, in a front view, the example design for the proximal end of the secondary shaft; and

FIG. 24D illustrates, in a left side view, the example design for the proximal end of the secondary shaft.

### DETAILED DESCRIPTION

For illustrative purposes, specific example embodiments will now be explained in greater detail in conjunction with the figures.

The embodiments set forth herein represent information sufficient to practice the claimed subject matter and illustrate ways of practicing such subject matter. Upon reading the following description in light of the accompanying figures, those of skill in the art will understand the concepts of the claimed subject matter and will recognize applications of these concepts not particularly addressed herein. It should be understood that these concepts and applications fall within the scope of the disclosure and the accompanying claims.

Moreover, it will be appreciated that any module, component, or device disclosed herein that executes instructions may include, or otherwise have access to, a non-transitory computer/processor readable storage medium or media for storage of information, such as computer/processor readable instructions, data structures, program modules and/or other data. A non-exhaustive list of examples of non-transitory computer/processor readable storage media includes magnetic cassettes, magnetic tape, magnetic disk storage or other magnetic storage devices, optical disks such as compact disc read-only memory (CD-ROM), digital video discs or digital versatile discs (i.e., DVDs), Blu-ray Disc™, or other optical storage, volatile and non-volatile, removable and non-removable media implemented in any method or technology, random-access memory (RAM), read-only memory (ROM), electrically erasable programmable read-only memory (EEPROM), flash memory or other memory technology. Any such non-transitory computer/processor storage media may be part of a device/apparatus or accessible or connectable thereto. Computer/processor readable/executable instructions to implement a method, an application or a module described herein may be stored or otherwise held by such non-transitory computer/processor readable storage media.

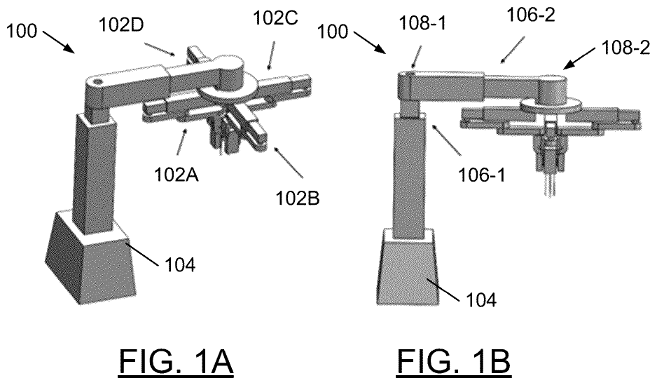

FIG. 1A illustrates, in a top-left perspective view, a surgical robot 100 according to aspects of the present disclosure. In FIG. 1A, the surgical robot 100 is illustrated as having four robotic arms: a first robotic arm 102A; a second robotic arm 102B; a third robotic arm 102C; and a fourth robotic arm 102D (collectively or individually 102). The four robotic arms 102 are attached to a single main platform 104.

FIG. 1B illustrates the surgical robot 100 in an elevation view. As illustrated in FIG. 1B, the main platform 104 has a plurality of joints, including: first prismatic joint 106-1; a second prismatic joint 106-2; a first main platform revolute joint 108-1; and a second main platform revolute joint 108-2. The main platform revolute joints (collectively or individually 108) may be shown to allow for positioning of the four robotic arms 102.

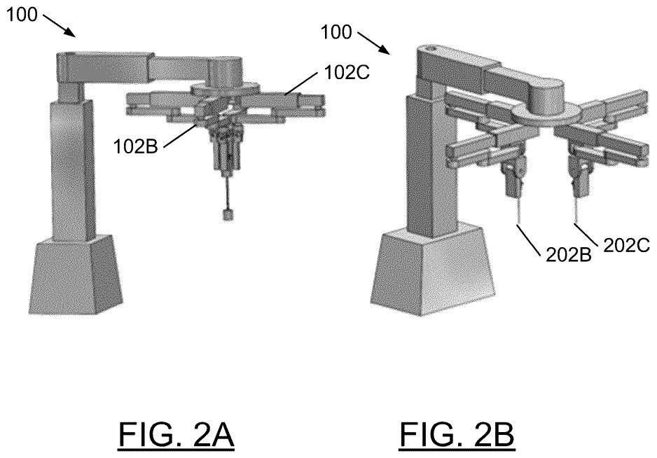

FIG. 2A illustrates the surgical robot 100 in a front-right perspective view. FIG. 2B illustrates the surgical robot 100 in a further front-right perspective view. In FIG. 2B, it may be observed that the second robotic arm 102B is associated with a second tool 202B and that the third robotic arm 102C is associated with a third tool 202C. It should be understood that, although not illustrated in FIG. 2B, the first robotic arm 102A is associated with a first tool and that the fourth robotic arm 102D is associated with a fourth tool. The tools may be associated, collectively or individually, with reference number 202. Each tool 202 may be associated with a tool shaft, which is not associated with a reference number.

The robotic arms 102 may be arranged in a first orientation. In the first orientation, illustrated in FIG. 2A, each tool 202 may be brought adjacent to each other in the middle along a single axis, with all four tool shafts fitting within a 1.5 cm diameter circle. The robotic arms 102 may be arranged in a second orientation. In the second orientation, illustrated in FIG. 2B, each tool 202 may be positioned away from each other, thereby allowing for triangulation.

The ability for the robotic arms 102 to be arranged in the first orientation (FIG. 2A) and in the second orientation (FIG. 2B) may be shown to allow for a transition between a single-port design and a multi-port design.

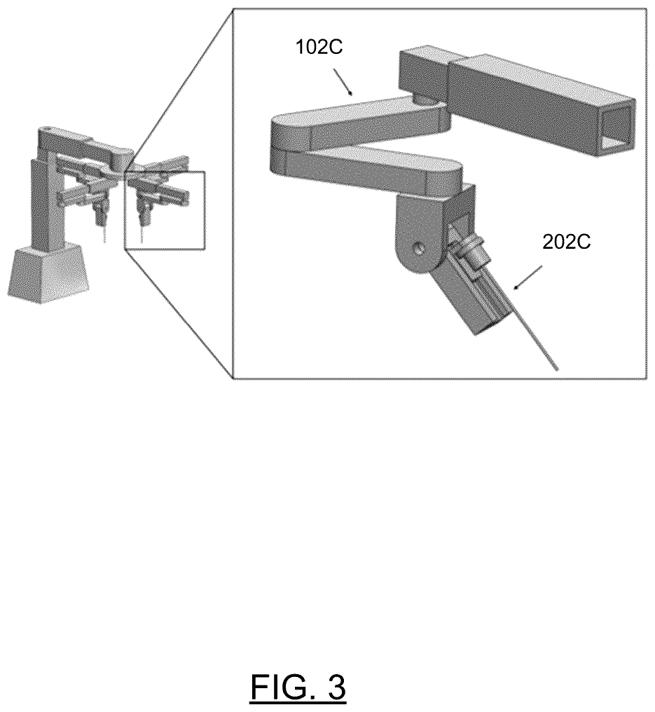

FIG. 3 illustrates the third robotic arm 102C with the third tool 202C. As illustrated in FIG. 3, the third tool 202C is detachable.

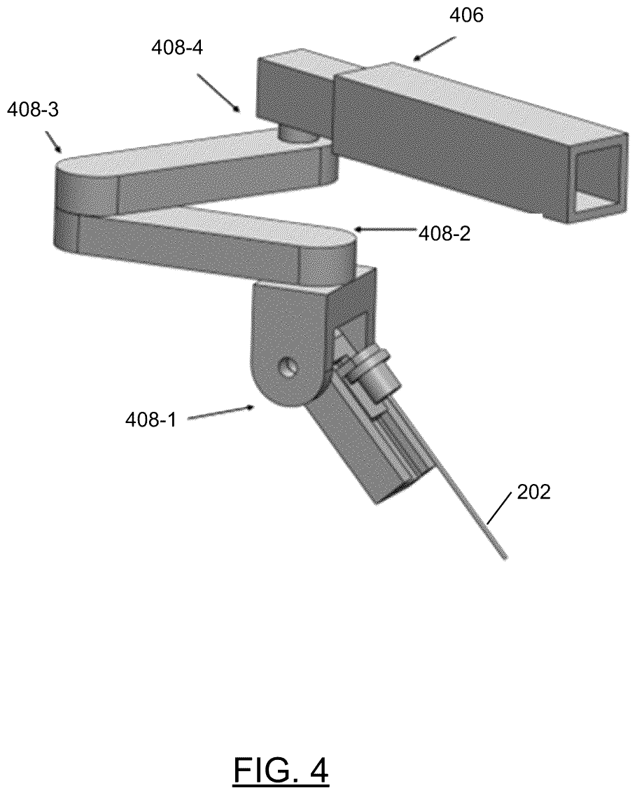

FIG. 4 illustrates one of the robotic arms 102, in isolation, to illustrate five setup joints and a tool 202. The setup joints include an arm prismatic joint 406 and four revolute joints. The four revolute joints include: a first arm revolute joint 408-1; a second arm revolute joint 408-2; a third arm revolute joint 408-3; and a fourth arm revolute joint 408-4 (collectively or individually 408).

The setup joints 406, 408 may be shown to allow positioning of the tool 202 in a plurality of positions and orientations.

Aspects of the present application relate to cases wherein the tool 202 at the end of the robotic arm 102 has six degrees of freedom. A joint configuration for the tool 202 may have a plurality of components. As will be described hereinafter, one component relates to a prismatic joint along the axis the tool shaft, another component relates to a roll joint, a further component relates to an elbow pitch joint, a still further component relates to an elbow yaw joint, an even further component relates to a wrist pitch joint and further components relate to a tooltip joints (yaw and jaw opening).

A major concern in developing cable-driven tools with multiple stages of joints is the behavior of the cables when proximal joints are actuated and the manner in which the cable behavior impacts actuation of distal joints. As will be described hereinafter, the design of the elbow joint (for pitch and yaw) accounts for two issues presented by the actuation of the elbow joint. A first issue relates to path length changes of a wrist cable and a tool tip cable. A second issue relates to cable interference.

Cable path length changes typically occur when there is deflection of a cable off an expected path in response to actuation of a joint. Such deflection occurs, most commonly, responsive to a point of contact of the cable in the joint not being on an axis of rotation of the joint. This may be shown to cause the cable to follow a path around a curvature of a joint bend. In this way, cables contacting on a concave side of a bent joint may take a shorter path relative to a path taken by the cable when the joint is in an unbent state. In contrast, cables contacting on a convex side of a bent joint may take a longer path relative to the path taken by the cable when the joint is in an unbent state. Path changes may be shown to prevent continuous cable tension. In one effect, a cable on the convex side lacks the slack to cover the new path. In another effect, a cable on the concave side has excess cable for the shorter path, thereby causing slack. Both effects may be shown to render the distal joints actuated by these cables non-functional. In aspects of the present application, there is an aim to counteract as much of the path length change mechanically to, thereby, account for remaining changes in controls. A pair of cables residing at some proximal joint may be associated with actuating one or more distal joints. According to aspects of the present disclosure, the pair of cables may be positioned such that the pair of cables belong to one of three categories: a first category, wherein both cables are positioned on the joint axis; a second category, wherein both cables are positioned on the same side of the joint axis; and a third category, wherein one cable is positioned on each side of the joint axis. Path length changes are insignificant for the first category and for the third category. If the contact point for the cables lies on the joint axis, no deflection occurs when the joint is bent, meaning there's no change in path length. If the contact points are on either side of the joint axis, the cables may simply be actuated in opposing directions, which is what the cable pair is already configured to do when the cable pair actuates the joint.

According to aspects of the present disclosure, the surgical robot will have as many cable pairings belonging to one of these categories as is practical. The locus of points through which the cables pass may be arranged such that a first elbow joint (pitch) is aligned with a first wrist joint (pitch) and the contacts of a second elbow joint (yaw) fall on an axis of the first elbow joint (pitch). Through this arrangement, the first elbow joint (pitch) does not create a path length change in the second elbow joint (yaw) or in the first wrist joint (pitch). Similarly, the second elbow joint (yaw) does not impact the first wrist joint (pitch). To achieve this arrangement, the cables may be arranged to pass through a center of two pin joints. The pin joints may be designed with holes for cables to pass through, with the contact point at the center of the pin joint or, in other words, with the contact point positioned along the joint axis. Due to the constraints of a tool shaft, tool tip cable pairings may be positioned off-center of both the pitch axis and the yaw axis. This positioning may be shown to mean that changes in the path length for tool tip cable pairings may be accounted for by independent control of each cable. Note that this issue already exists with non-elbowed tools. This will be addressed through the actuation module, discussed hereinafter.

Actuation of a proximal joint may also be shown to lead to changes in the path of a given cable in the proximal joint and the effects of these changes may include cable interference, particularly if points of contact are not fully constrained. While these effects do not typically come into play with wristed instruments, the density of cables in the tool shaft and added stage of joints in the elbow may cause cable-cable and cable-tool interactions to be a concern.

There are three main design components that serve to address this problem: the cable channel in the second link of the elbow; custom joint pins; and a custom roll joint. The first major issue is in the alignment of the first elbow joint (pitch) and the first wrist joint (pitch), which may be shown to lie on the same axis. When the first elbow joint (pitch) is actuated, the point of contact of the cables may move circularly about the joint axis. Without further adjustment of the cable path, the cable on the convex side of the joint forms a diagonal to the new position of the point of contact. Because the first elbow and first wrist joint lie on the same axis, there may be interference between cables as the joint angle becomes more extreme. To prevent this interference, the cable may be redirected along a circular path that may be shown to maintain a consistent contact point location on the convex side of the joint. This circular path may be maintained by a track on a second link of the elbow. The cables actuating the first elbow joint (pitch) may have a diameter that is approximately double the diameter of the cables actuating the other joints. The track may be designed such that the elbow joint cables run along the track while allowing the wrist cables to pass through the center of the track unaffected. Cable-cable interference in the other cables is prevented mainly through the design of the pins, which fixes the location of the contact point of the cables. The other cables may not be aligned in a way that would cause cable-cable interference at extreme angles, so additional adjustment of their paths may be safely ignored. However, to ensure easier calculation for control and consistent cable positioning entering distal joints, the distal side of the pin may be designed as a half circle to consistently direct the cables into a locus matching the expected arrangement at the wrist through a pre-calculated circular path. The circular path also prevents the cable from going around a sudden corner, which could potentially cause increased friction.

Lastly, actuation of the proximal roll joint may lead to twisting of the cables, which could significantly affect cable movement at extreme roll values. Twisting occurs because the distal end of the tool rotates, while the actuation end remains static. The twisting may be addressed through a mechanism positioned directly at the roll joint. The mechanism may allow the cables to twist around a smooth tube that prevents the cables from taking the shortest path across the cross section of the tube. Indeed, this mechanism may be shown to constrain the twisting to a specified area to, thereby, prevent cable-cable interference within this area.

To enable single-port-like capabilities in a TORS system built according to aspects of the present disclosure, a profile of tool actuation may be manipulated such that the tool shaft centers may be positioned as close as is practical. This may be shown to be extremely challenging to achieve when actuation uses capstans and pulleys, which generally involve use of significant space perpendicular to the axis of the tool shaft. The space largely comes from the space that would, otherwise, be used to redirect the cables. This challenge is increased responsive to the addition of further degrees of freedom.

Aspects of the present disclosure relate to a lead-screw-based tool actuator (not shown). The lead-screw-based actuator may be shown to allow for a tool actuation structure to be elongated along the axis of the tool shaft, rather than necessitating the lateral expansion that separates the tool shafts. Tubes connected to the cables may be attached to a custom slider attached to a nut mounted on a lead screw. When the lead screw is rotated, the nut moves linearly, pulling or releasing the cable in a desired direction. The slider attached to the nut may be shaped according to a determined profile without significantly impacting the effectiveness of the actuation, assuming that it is possible to include a guiding rail constraining the rotation of the slider about the axis of the lead screw. This allows the profile of the tool actuation structure to be easily manipulable, thus enabling a triangular structure that allows for single-port-like arrangements of four tools 202 at the ends of the four respective robotic arms 102. The tool actuation structure may, itself, be divided into two stages, one stage for elbow joints and one stage for wrist joints and tool tip joints. This division of the tool actuation structure into two stages may be shown to prevent interference between the sliders connected to wrist cables lying on an inner layer of the locus and those sliders connected to the elbow cables lying on the outer layer of the locus. The first stage contains elbow joint sliders, the lead screws for which pass through the entire length of the two stages. The lead screws for the second stage only pass through the second stage. All lead screws interface to an actuation module. The actuation module may be implemented as a 6 mm diameter motor supplied, for example, by Maxon International Ltd. of Sachseln, Switzerland.

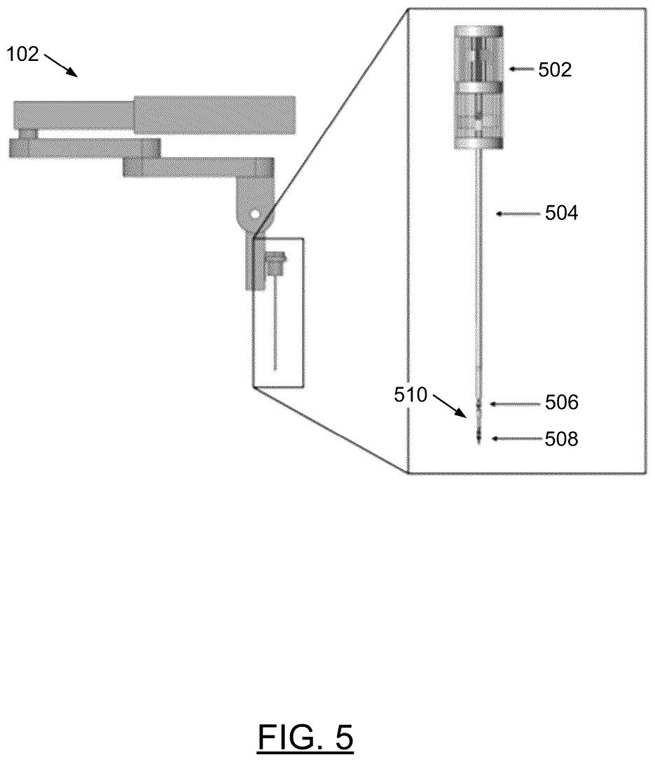

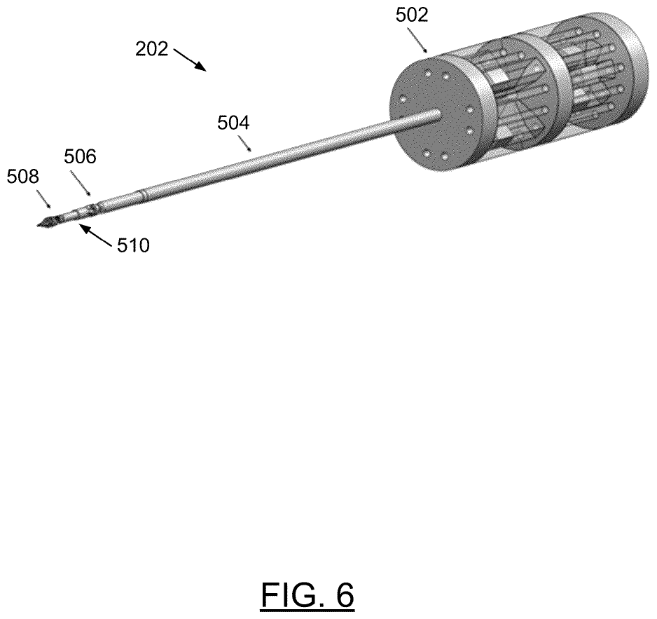

FIG. 5 illustrates, in an elevation view, a generic one of the tools 202 associated with a generic one of the robotic arms 102. FIG. 6 illustrates, in a perspective view, the tool 202 of FIG. 5. The tool 202 is illustrated, in FIG. 5 and FIG. 6, as including a proximal driving unit 502, a tool shaft 504, an elbow joint 506, a secondary shaft 510 and a wrist joint 508. The driving unit 502 may be actuated by the robotic arm 102 at a coupling mechanism (not shown).

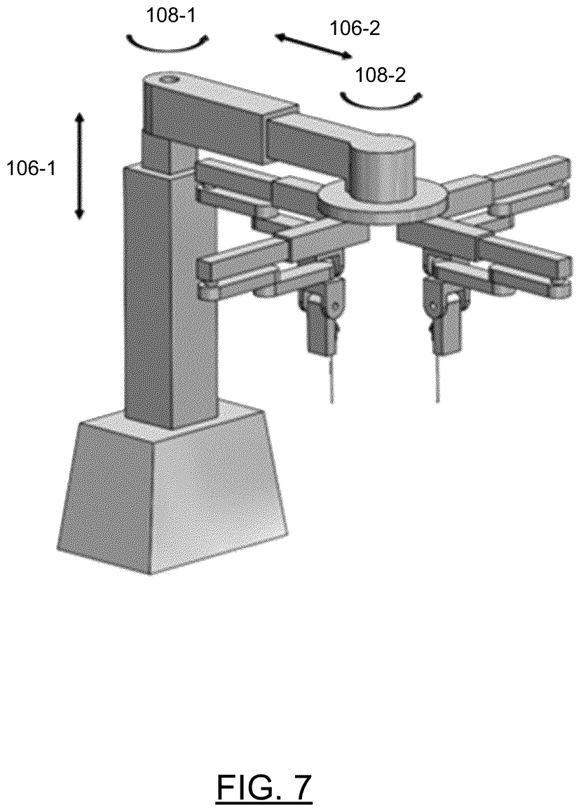

Positioning and orienting the tools 202 for a desired position and pose may be accomplished using the main platform joints and the robotic arm joints. FIG. 7 illustrates the DOF and motion of the main platform joints, including the first prismatic joint 106-1, the second prismatic joint 106-2, the first main platform revolute joint 108 and the second main platform revolute joint 108-2. The main platform revolute joints 108 may be shown to allow for positioning of the four robotic arms 102.

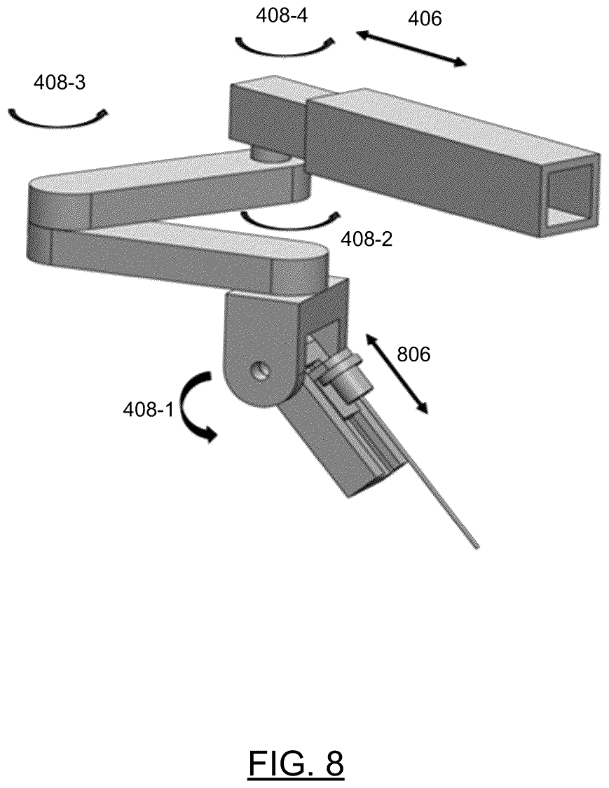

FIG. 8 illustrates the DOF and motion of the robotic arm joints, including the arm prismatic joint 406, the first arm revolute joint 408-1, the second arm revolute joint 408-2, the third arm revolute joint 408-3, the fourth arm revolute joint 408-4 and a tool prismatic joint 806.

The tool shaft 504 provides length to the tool 202. The elbow joint 506 has at least two DOF (selected from, say, elbow roll, elbow pitch and elbow yaw) The wrist joint 508 has at least two DOF (selected from, say, wrist pitch, wrist yaw and wrist grip) The wrist joint 508 may be fitted with an end-effector.

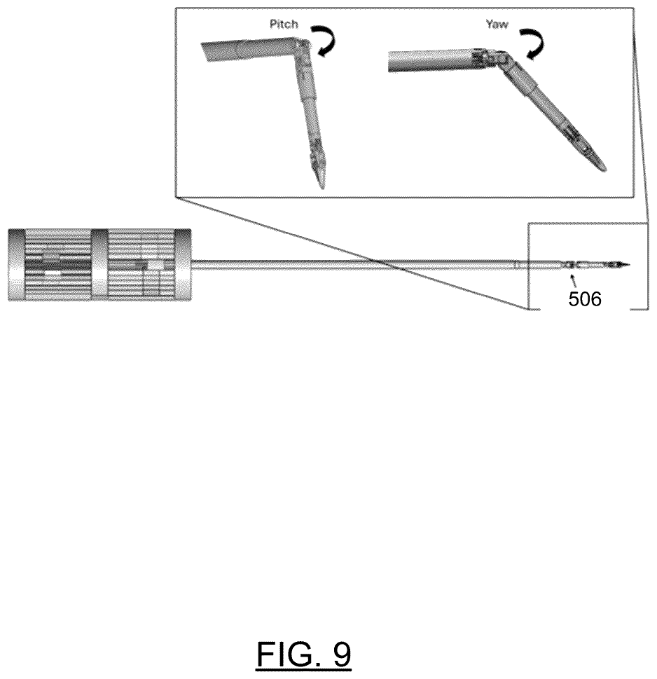

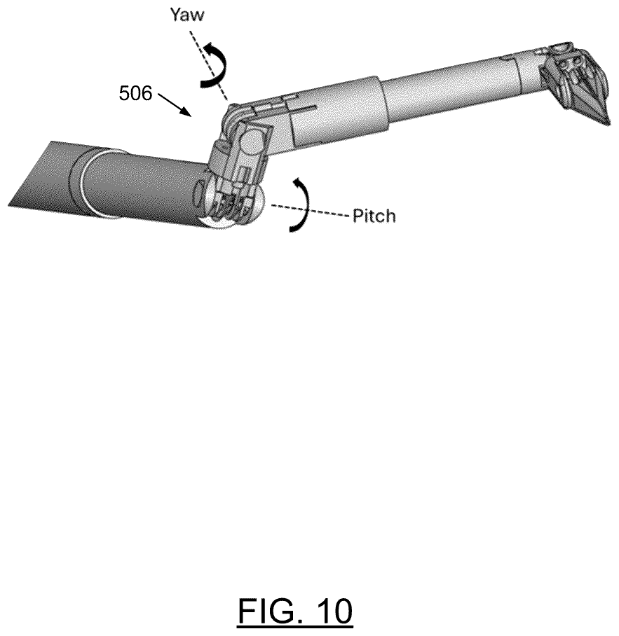

The elbow joint 506 was developed as part of the tool to provide additional DOF to allow the end-effector improved motion, reachability and dexterity within constrained environments while reducing instrument collisions. The elbow joint 506 may be implemented as a joint with at least two degrees-of-freedom, as illustrated in FIG. 9. The two-DOF elbow joint 506 may have an elbow yaw function and an elbow pitch function controlled by respective revolute joints, as illustrated in FIG. 10.

The wrist joint 508 may be implemented as a pin jointed mechanism that includes four links and three DOF (pitch, yaw and grip). Furthermore, the secondary shaft 510 may be shown to provide the wrist joint 508 with a roll DOF.

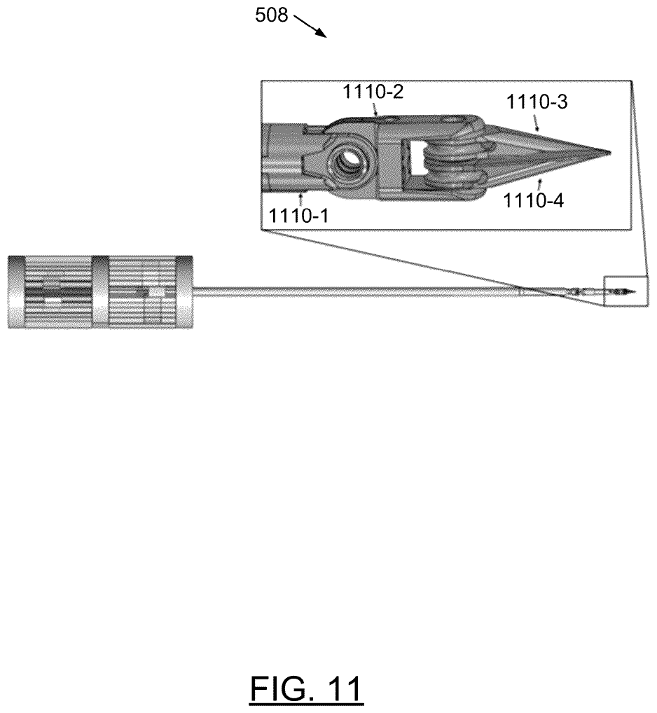

FIG. 11 illustrates four links of the wrist joint 508, including two main links, a first link 1110-1 and a second link 1110-2, as well as two subsidiary links, including a third link 1110-3 and a fourth link 1110-4. The third link 1110-3 and the fourth link 1110-4 may be described as tines that provide simultaneous yaw and grip function. The tines 1110-3, 1110-4 may be implemented as different tool-tips, such as, for only three examples, scissors, graspers or needle drivers.

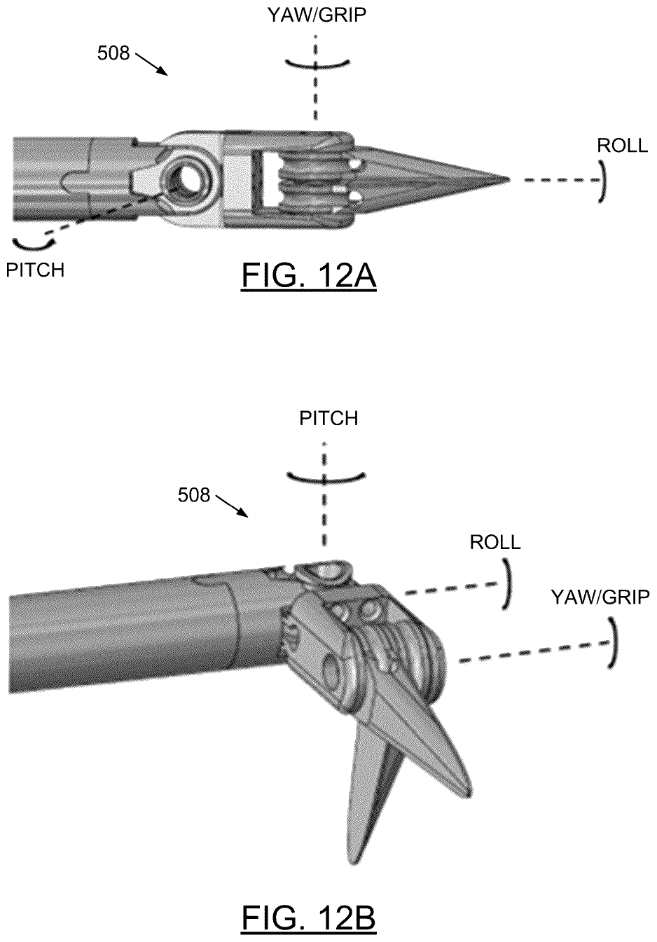

FIG. 12A illustrates the DOF and joint axis of the wrist joint 508 including a roll function, a pitch function, a yaw function and a grip function.

FIG. 12B illustrates the wrist joint 508 in an example pose, demonstrating the pitch function and the yaw/grip function.

The tool 202 described hereinbefore may be particularly well-suited to use in conjunction with the surgical robot 100, with the capability of achieving a single-port-like orientation through a port with 1.5 cm diameter.

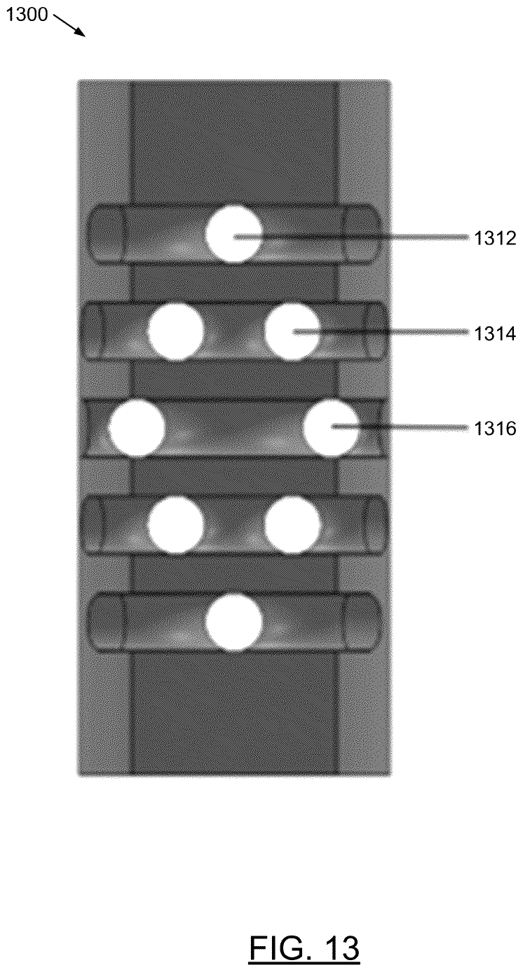

FIG. 13 illustrates, in a side view, a custom pin 1300 for the elbow joint 506 (pitch). The custom pin 1300 includes a first passage 1312 for a cable for actuating the elbow joint 506 (yaw), a second passage 1314 for a cable for actuating the tool tip and a third passage 1316 for a cable for actuating the wrist joint 508 (pitch).

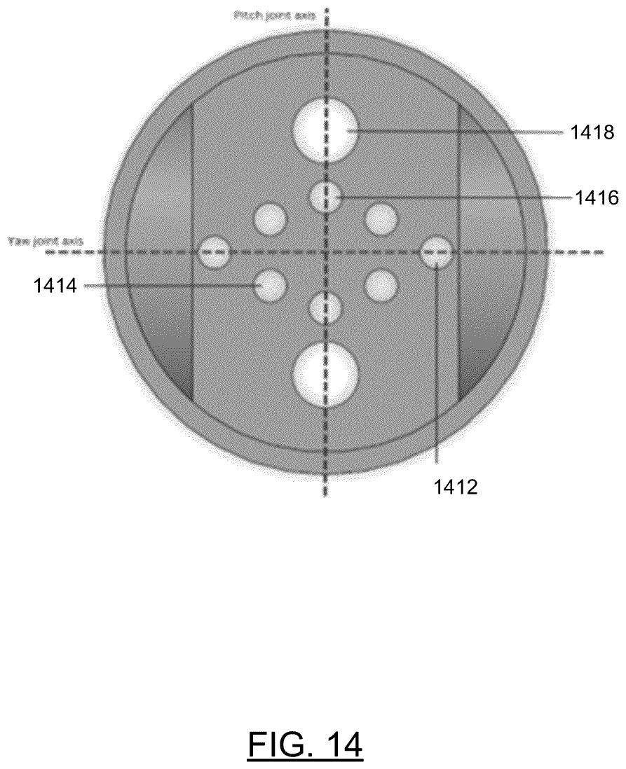

FIG. 14 illustrates cables prior to entering the first elbow joint 506 (pitch). The cables include a first cable 1412 for actuating the elbow joint 506 (yaw), a second cable 1414 for actuating the tool tip, a third cable 1416 for actuating the wrist joint 508 (pitch) and a fourth cable 1418 for actuating the first elbow joint 506 (pitch). Notably, any antagonistic cable pairing falling on the labelled joint axes is not expected to exhibit significant path length changes.

Development of the tool 202 has presented four major challenges: cable path length change; cable interference; cable twisting in extreme roll configurations; and enlarged tool profiles. Aspects of the present disclosure address the issue of cable path length change by passing cables through custom pins (such as the custom pin 1300 of FIG. 13). The custom pins may position contact points of the cables at each joint such that antagonistic cable pairings either fall directly on the joint axis or fall on opposite sides of the joint axis (see FIG. 14).

The custom pins may reduce the number of cables for which cable path length is to be adjusted for on the actuation and controls end, thereby resulting in more robust control of the tool 202 in extreme positions. Furthermore, rigid contact points may be shown to prevent unpredictable cable pathing that leads to cable-cable interference. A second link (see FIG. 10) of the elbow joint 506 may also be designed to minimize interference between the cables responsible for pitch motion at the elbow joint 506 and the cables responsible for pitch motion at the wrist joint 508. The cables would otherwise interfere at greater bend angles of the elbow joint 506 because the cables are aligned. The interference minimization may be achieved through a track that forces the elbow pitch cables 1418 to sit along an arc about the joint axis while the wrist pitch cables 1416 are allowed to pass through unobstructed.

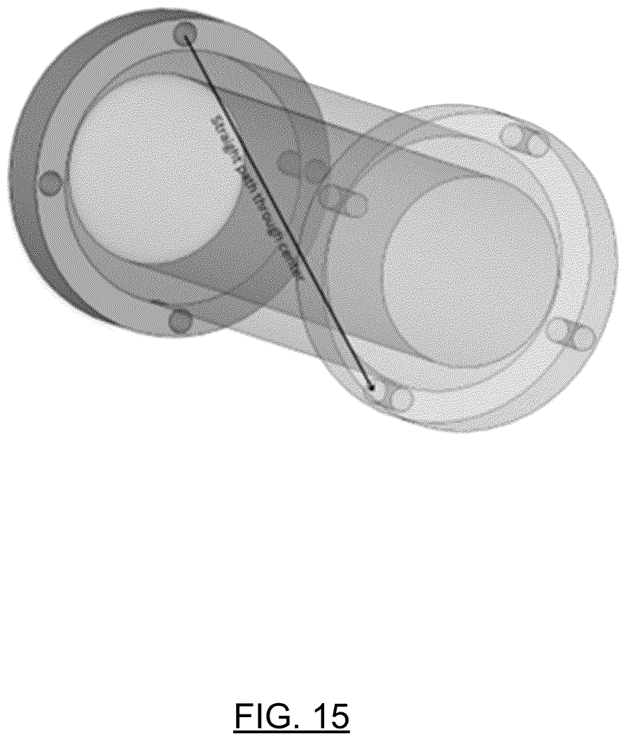

The cables also twist because of the roll function carried out at the wrist joint 508. The roll function may cause cables to tangle at the center of the tool 202 (see FIG. 15) when the tool 202 is rolled significantly, an effect exacerbated by the increased cable density in the elbowed tool 202.

FIG. 15 illustrates a path of a cable actuating the elbow joint 506 at a ˜135 degrees roll with no tube.

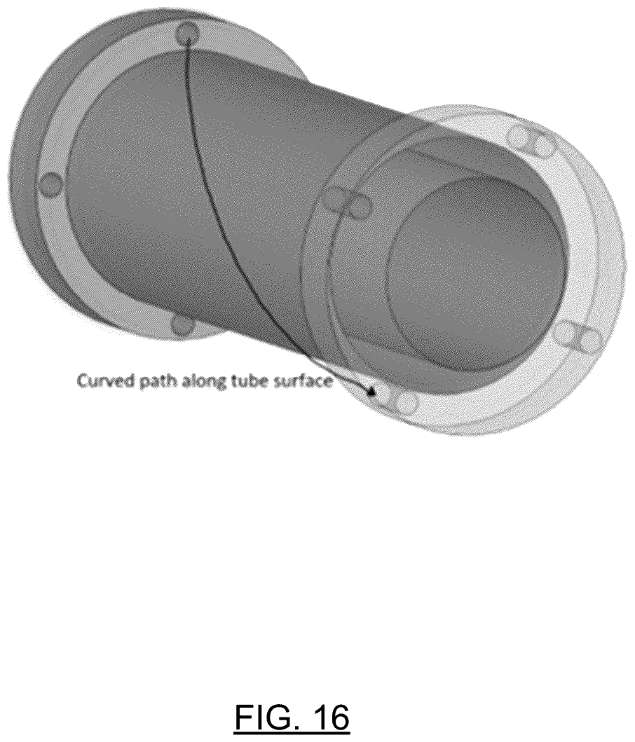

Aspects of the present disclosure address this issue by isolating the twisting of the cables to a proximal region where a tube redirects the path of the cables in a helical shape (see FIG. 16), thereby preventing the cables from interfering near the center of the tool shaft 504.

FIG. 16 illustrates a path of a cable actuating the elbow joint 506, wherein the cable wraps around the tube at a ˜135 degrees roll.

To achieve single-port-like arrangements, the distance between centers of tool shafts 504 is to be minimized. Existing tools use capstans and pulley systems to actuate. However, the space that cable pathing takes makes it challenging to reduce the size of the tool profile using this actuator. Aspects of the present disclosure involve using a lead-screw-based actuator instead of a capstan and pulley system. The lead-screw-based actuator may be shown to allow the tool actuation structure to be manipulated into a desired profile, such as a profile that enables the desired single port configuration.

Manipulating the tool actuation structure into a desired profile may be achieved using a two-stage slider system that connects into cables of the two joint stages. Each slider contains a nut. The nut may be moved along a corresponding lead screw to actuate a corresponding joint. Each lead screw may be actuated by a corresponding motor. Using the specified triangular profile for each tool 202, it may be shown that four tools 202 may be contained within a 1.5 cm diameter port.

The design of the robotic arms 102 allows the robotic arms 102 and corresponding tools 202 to be positioned for use as either a multi-port robot or a single-port robot.

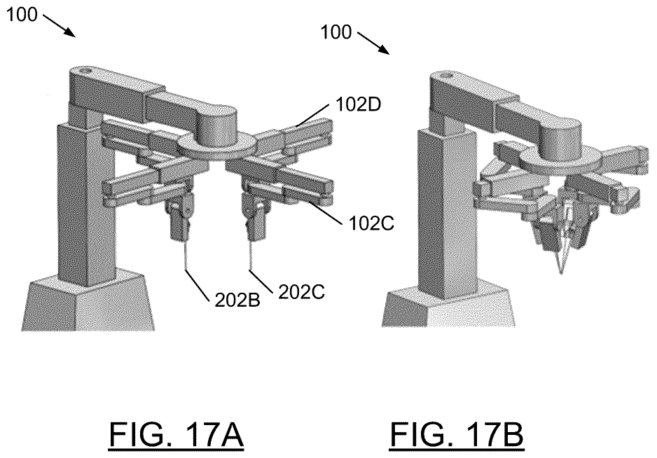

The robotic arms 102 may be positioned to allow for separation of the robotic arms 102 with triangulation of the robotic arms 102 towards an operative site. The separation of the tools 202 represents a multi-port orientation that may be beneficial for large body cavity surgery, including pelvic surgical procedures, abdominal surgical procedures and thoracic surgical procedures. FIG. 17A illustrates a multi-port setup of the robot.

FIG. 17A illustrates a multi-port robotic arm 102 positioning wherein each robotic arm 102 and tool 202 are separated from each other.

FIG. 17B illustrates a typical multi-port setup with tools 202 triangulating towards a common point. The common point may be understood to be representative of an operative site. Entrance into a body cavity sometimes involves use of multiple ports through a body cavity wall, such as at the pelvis, at the abdomen or at the thorax.

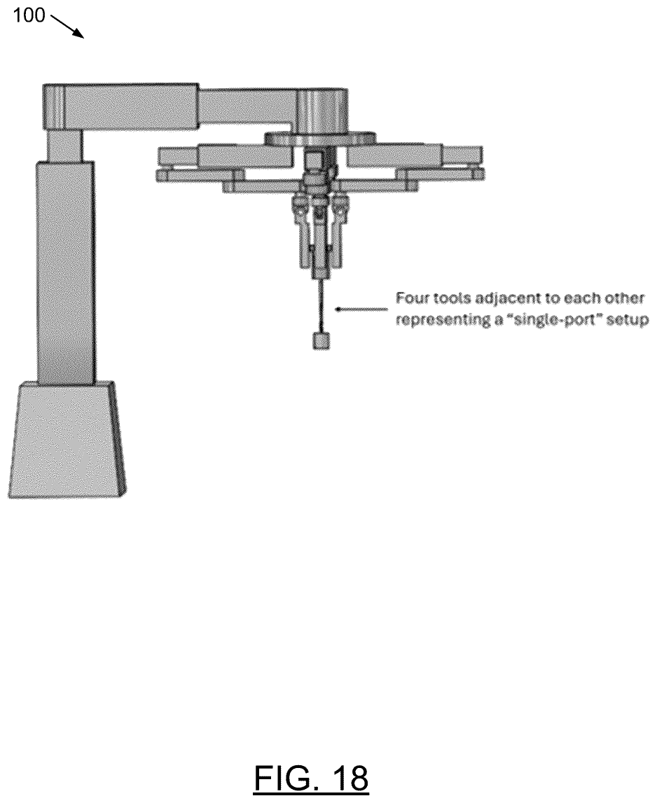

Aspects of the present disclosure allow for orientation of the tools 202 such that the surgical robot 100 may operate as a single-port system. FIG. 18 illustrates the robotic arms 102 and tools 202 adjacent to each other with the tool shafts 504 parallel in very close proximity allowing the tool shafts 504 to traverse through a single port or incision through a body cavity wall. The elbow joints 506 allow for motion at the tool tips within small workspace areas.

FIG. 18 illustrates a “single-port” setup wherein all four tools 202 are positioned adjacent to each other in a parallel orientation. In the single-port setup, all tools 202 can traverse through a single trocar and incision of a body cavity, such as the pelvis, the abdomen or the thorax.

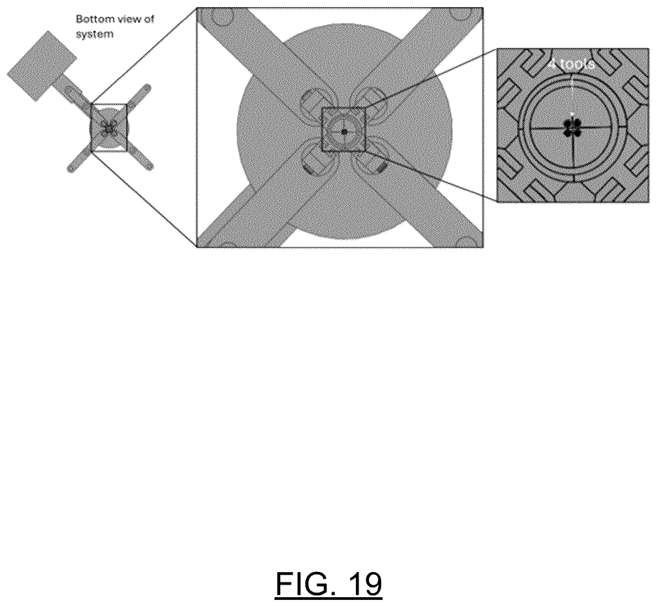

FIG. 19 illustrates an axial view from below illustrating the robotic arms 102 and the tools 202 adjacent to each other with minimal clearance from each other. This allows all tools to traverse through a single trocar and incision.

It should be clear, to a person of ordinary skill, that the descriptions of the surgical robot 100 in the present disclosure represent only one embodiment of the surgical robot 100. There can be multiple different embodiments of the design that can accomplish the same function.

Conveniently, the surgical robot 100 has a setup joint mechanism to position and orient multiple tools. The setup joints can have any configuration and any number of DOF. The surgical robot 100 may function as either a single-port or multi-port system. The tool 202 that attaches to the setup joints includes an elbow joint and a wrist joint. The tool 202 may have any configuration and any number of DOF. The elbow joint can have any configuration and any number of DOF. The wrist joint can have any configuration and any number of DOF. Indeed, the surgical robot 100 may also have a design that is more compact than described herein. A more compact design may, for example, be accomplished using smaller setup joints.

Aspects of the present disclosure relate to a distal compact wrist joint with at least two DOF (selected from wrist pitch, wrist yaw and wrist grip) for fine tip manipulation. The distal compact wrist may be implemented with a 3 mm diameter.

Aspects of the present disclosure relate to a proximal elbow joint with at least two DOF (pitch, yaw), allowing for improved wrist positioning and orientation within the oropharyngeal cavity. With the tool shaft 504 providing, to the elbow joint 506, a roll DOF, it may be shown that the elbow-wrist tool 202 may operate with as many as six DOF.

Aspects of the present disclosure relate to utilizing revolute joints at all DOF to maximize the compactness of articulation at the elbow joint and the wrist joint, thereby allowing for improved performance within confined workspaces.

Aspects of the present disclosure relate to choosing the distance between the elbow joint and the wrist joint to allow for compact articulation within the oropharyngeal space.

Aspects of the present disclosure relate to coupling the elbow-wrist tool 202 to a 7-DOF manipulator to provide roll and translation of the proximal shaft.

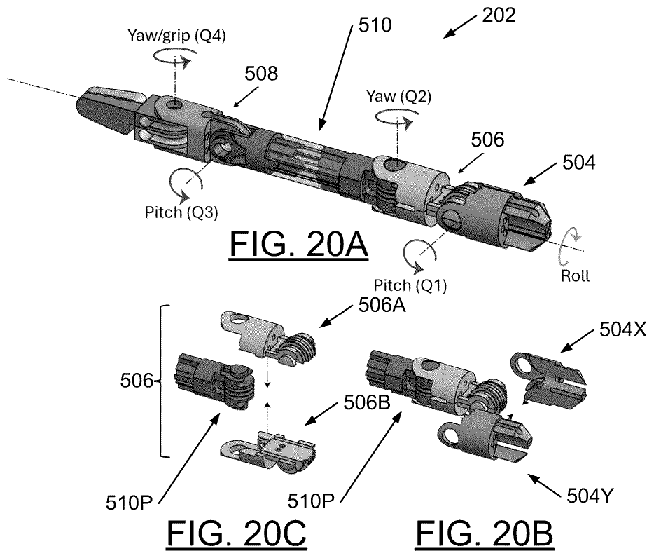

FIG. 20A schematically illustrates the elbow-wrist tool 202 with the elbow joint 506 and the wrist joint 508 associated with an axis for each respective degree-of-freedom. FIG. 20B illustrates, in an exploded view, the elbow joint 506 with the sandwich design that eliminates the need for pins and allows for routing of tendons (cables) for elbow motion and wrist motion. FIG. 20C illustrates, in an exploded view, another aspect of the sandwich design of the elbow joint 506. FIG. 20A illustrates the elbow-wrist tool 202, including the tool shaft 504, the elbow joint 506, the secondary shaft 510 and the wrist joint 508. FIG. 20B, in particular, emphasizes the manner in which the elbow joint 506 connects to a proximal end 510P of the secondary shaft 510. The exploded view of FIG. 20B clarifies that the sandwich design of the elbow joint 506 includes a first elbow joint part 506A and a second elbow joint part 506B. FIG. 20C, in particular, emphasizes the manner in which the elbow joint 506 connects to the tool shaft 504. The exploded view of FIG. 20C clarifies that the sandwich design of the elbow joint 506 includes a first tool shaft part 504X and a second tool shaft part 504Y.

Miniaturizing a tendon-driven elbow may be shown to involve routing of eight tendons through two proximal revolute joints while (i) isolating antagonistic pairs, (ii) respecting the minimum bending radius of the cable and (iii) limiting path-length changes to minimize coupling. Aspects of the present disclosure relate to a two-piece “sandwich” link architecture, where the two proximal links are split, providing access to internal planes of the joint for machining cable guide channels (see FIG. 20A, FIG. 20B and FIG. 20C). Such a link architecture may also be shown to enable a pin-less assembly in which rotation occurs about a bushing machined into the halves, thereby reducing parts and easing assembly. Links may be machined from stainless steel. Machining constraints may be understood to impose a 0.5-mm minimum through-hole that, in turn, sets a feasible tendon size. Of course, this is a present-day constraint and should not be considered as limiting the tendon size of future implementations of aspects of the present disclosure. For an example tendon, a 0.24-mm diameter, 7-strand braided tungsten cable (from Carl Stahl Sava Industries Inc., Riverdale, NJ, USA) may be utilized across the elbow joint 506 and the wrist joint 508. Conveniently, an implementation utilizing this example tendon may be shown to maximize strength and joint rotation without increasing link diameter. Alternatively, a cable formed of stainless steel or nylon, or any plastic or metal may be used.

Aspects of the present disclosure relate to using circular channels and spiral channels for cable routing. The circular channels may be used to offset cables about an axis when geometry prohibits centerline routing. The spiral channels may be used when geometry allows routing through (or very near) the rotational axis. Spiral channels may be shown to respect cable bend limits while minimizing path-length variation.

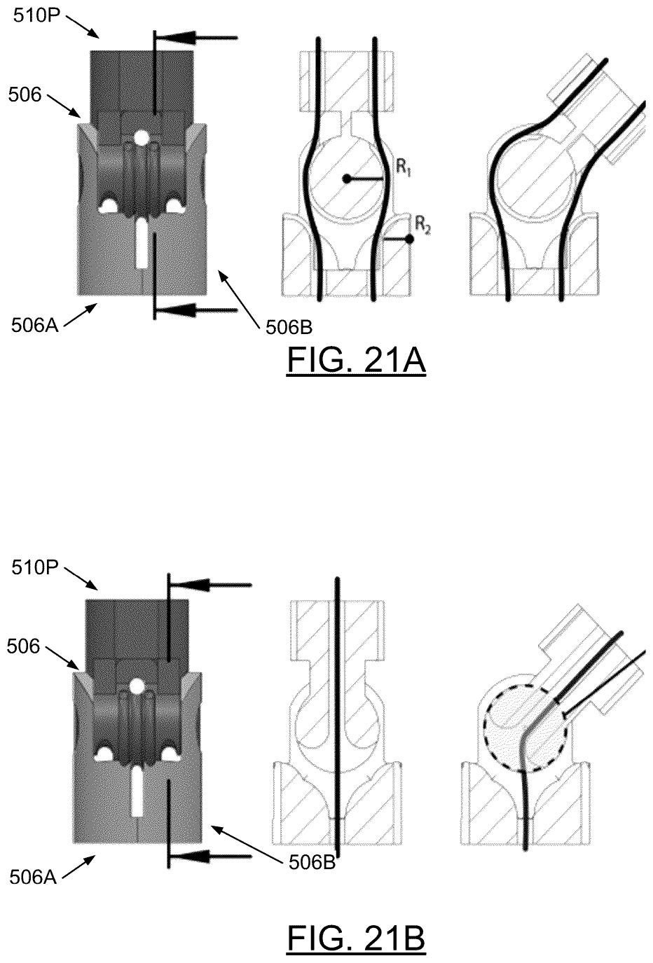

FIG. 21A and FIG. 21B illustrate example cable routing for the elbow joint 506. In FIG. 21A, the elbow joint 506 features circular grooves. In FIG. 21B, the elbow joint 506 features spiral grooves. In FIG. 21A, the elbow joint 506 is illustrated in a pair of section views: a section view in which the proximal end 510P of the secondary shaft 510 is aligned with the combination of the first elbow joint part 506A and the second elbow joint part 506B; and a section view in which the proximal end 510P of the secondary shaft 510 has an angular offset relative to the combination of the first elbow joint part 506A and the second elbow joint part 506B. Similarly, in FIG. 21B, the elbow joint 506 is illustrated in a pair of section views: a section view in which the proximal end 510P of the secondary shaft 510 is aligned with the combination of the first elbow joint part 506A and the second elbow joint part 506B; and a section view in which the proximal end 510P of the secondary shaft 510 has an angular offset relative to the combination of the first elbow joint part 506A and the second elbow joint part 506B.

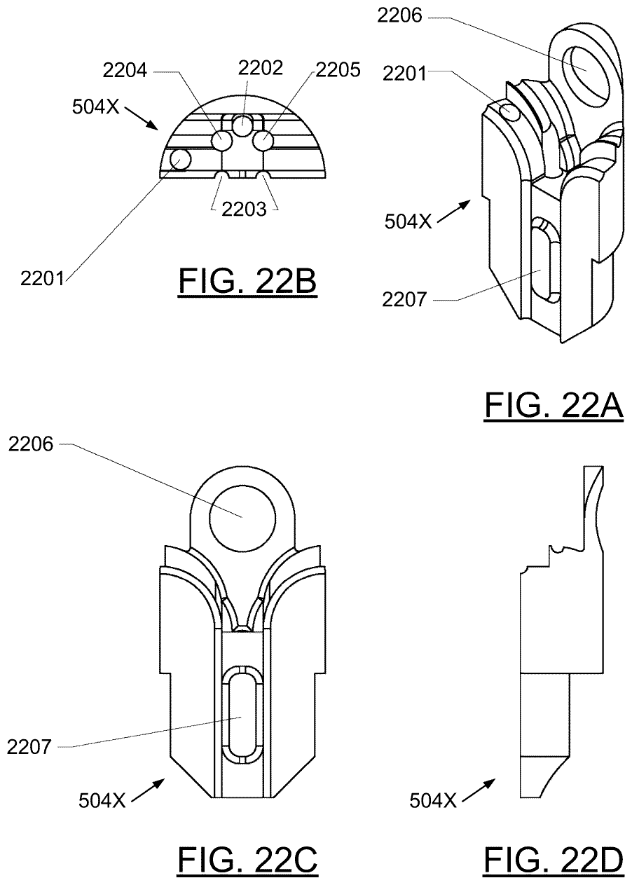

FIG. 22A illustrates, in a right, front isometric view, the example design for the first tool shaft part 504X. FIG. 22B illustrates, in a top plan view, an example design for the first tool shaft part 504X. FIG. 22C illustrates, in a front view, the example design for the first tool shaft part 504X. FIG. 22D illustrates, in a right side view, the example design for the first tool shaft part 504X. The first tool shaft part 504X in these figures includes a cable guide channel 2201 for a second link of the elbow joint 506, a cable guide channel 2202 for a third link of the elbow joint 506, a cable guide channel 2203 for a second link of the wrist joint 508, a cable guide channel 2204 for a right jaw of a gripper mechanism illustrated at the distal end of the tool 202 in FIG. 20A, a cable guide channel 2205 for a left jaw of the gripper mechanism, a joint location 2206 of the link that enables rotation and a divider feature 2207 to divide antagonistic cables of the second link of the wrist joint 508.

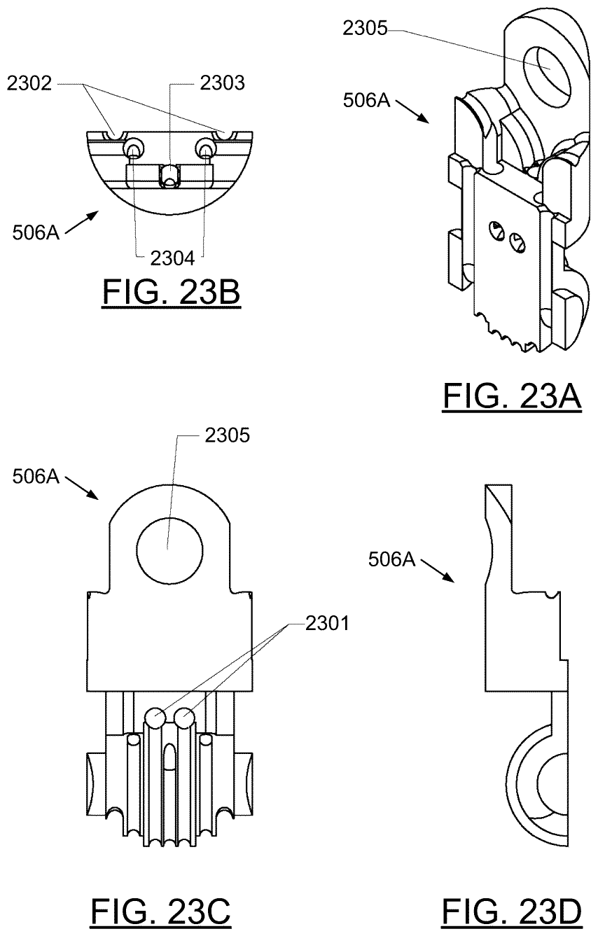

FIG. 23A illustrates, in a right, front isometric view, the example design for the first elbow joint part 506A. FIG. 23B illustrates, in a top plan view, an example design for the first elbow joint part 506A. FIG. 23C illustrates, in a front view, the example design for the first elbow joint part 506A. FIG. 23D illustrates, in a left side view, the example design for the first elbow joint part 506A. The first elbow joint part 506A in these figures includes a cable guide channel 2301 for the second link of the elbow joint 506, a cable guide channel 2302 for the third link of the elbow joint 506, a cable guide channel 2303 for the second link of the wrist joint 508, a cable guide channel 2304 for the left jaw of the gripper mechanism and a joint location 2305 of the link that enables rotation.

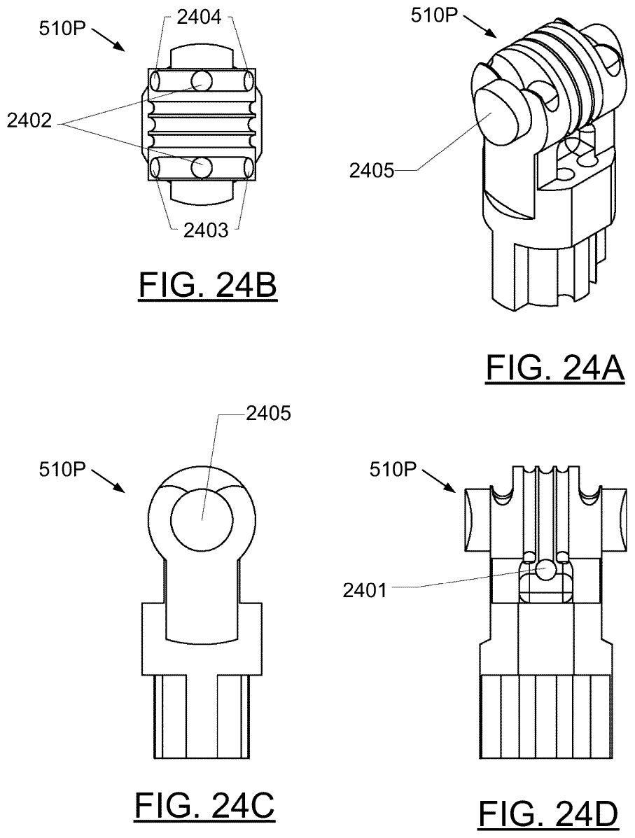

FIG. 24A illustrates, in a right, front isometric view, the example design for the proximal end 510P of the secondary shaft 510. FIG. 24B illustrates, in a top plan view, an example design for the proximal end 510P of the secondary shaft 510. FIG. 24C illustrates, in a front view, the example design for the proximal end 510P of the secondary shaft 510. FIG. 24D illustrates, in a left side view, the example design for the proximal end 510P of the secondary shaft 510. The proximal end 510P of the secondary shaft 510 in these figures includes a cable guide channel 2401 for the third link of the elbow joint 506, a cable guide channel 2402 for the second link of the wrist joint 508, a cable guide channel 2403 for the right jaw of the gripper mechanism, a cable guide channel 2404 for the left jaw of the gripper mechanism and a joint location 2405 of the link that enables rotation.

To summarize the design of the wrist joint 508, the wrist joint 508 comprises at least two DOF (selected from wrist pitch, wrist yaw and wrist grip) and may be implemented with a 3-mm diameter. The wrist joint 508 comprises four links with cable guide channels for cable routing that minimizes link length, resulting in compact articulation. Braided tungsten tendons (for example, 0.24 mm tendons) may be routed through the wrist joint 508 for control of a pitch function, control of a yaw function and control of a grip function. A proximal cam mechanism may be utilized to mechanically decouple the pitch function of the wrist joint 508 from the yaw/grip function of the wrist joint 508. Antagonistic tendons for each DOF may be controlled by separate actuators, thereby eliminating the use for a mechanical decoupling mechanism.

A length for the secondary shaft 510, which defines a distance between the elbow joint 506 and the wrist joint 508, may be determined by spatial constraints associated with the infant oral cavity, as evaluated using a high-fidelity cleft palate simulator. A 2.2-cm length for the secondary shaft 510 may, for example, be selected, thereby permitting full articulation of the elbow joint 506 and full articulation of the wrist joint 508 within the infant oral cavity. This length for the secondary shaft 510 parameter, however, is adaptable and can be lengthened or shortened depending on the requirements of specific applications.

The proximal driving unit 502 (also referred to as an actuation box) may employ Dynamixel actuators (from ROBOTIS Co. Ltd. of Seoul, South Korea) to tension the tendons and move the tendons. The tendons for the elbow joint 506 may be driven by capstans. The tendons for the wrist joint 508 may be driven by lead screws to enable independent antagonistic pre-tensioning and closed-loop differential control, which may be shown to compensate for tensile coupling. The tool 202 may be mounted on a Franka Research 3 robotic arm from (Franka Robotics of Munich, Germany), to provides positioning and tool-shaft roll and translation. Coordinate conventions may be fixed so that base-frame roll is clockwise as viewed from the main platform 104, which is illustrated in FIG. 1.

Forward joint kinematics of the elbow-wrist instrument (the tool 202) may be understood to involve investigating the cable routing and motor-to-joint space kinematics.

It may be understood that the use of guide channels for cable routing introduces mechanical coupling, where motion at a proximal (upstream) joint unintentionally affects more distal (downstream) joints. The addition of redundant elbow joints into the design increases the number of coupled joint interactions. The coupled joint interactions may be analyzed and compensated for to, thereby, enable precise robot end-effector control. The coupled joint interactions may be categorized into kinematic coupling interactions and tensile coupling interactions. The categorizing may be carried out on the basis of the effect on the antagonistic cable pair of a downstream joint.

Kinematic coupling induces unintended motion. Kinematic coupling may be shown to occur when rotation of an upstream (proximal) joint causes a differential change in the antagonistic cable lengths of an downstream joint. This kinematic coupling may be shown to be a result of routing antagonistic cables on opposite sides of the axis of rotation.

Tensile coupling may be shown to affect cable tension. Tensile coupling may be shown to occur when the change in cable length of an antagonistic pair is symmetrical. This may be shown to be a result of routing both cables through symmetrical channels on the same side of the axis of rotation, thereby resulting in identical cable path length change as the joint actuates.

Identifying and quantifying the dependencies of each downstream link may be shown to assist in compensating for these coupling effects. Spiral guided channels drive coupling between adjacent links. In the ideal scenario, a cable passing through the rotational axis would experience no path length change as the joint moves, resulting in zero coupling.

The proximal driving unit 502 may include a processor (not shown) adapted to implement a real-time compensation strategy to counteract the kinematic relationships outlined hereinbefore. The processor may execute an algorithm configured to implement the real-time compensation strategy by actively counteracting the coupling-induced cable length changes. For a given proximal motion of a k^{th} joint angle, qₖ, k∈{1,2}, affecting an n^{th} distal joint, where n∈{3,4R, 4L}, the algorithm may be considered to have three steps: determine a coupling effect; determine a motor command angle; and apply real-time compensation to the motor command angle.

To determine the coupling effect, an induced cable length difference, Δl_{coupling}, may be determined, as follows:

$$Δ {l}_{coupling} = [ {l}^{+} ( k + 2 , {q}_{k} ) - {l}^{-} ( k + 2 , {q}_{k} ) ] - [ {l}^{+} ( k + 2 , 0 ) - {l}^{-} ( k + 2 , 0 ) ] , k ∈ \{ 1 , 2 \}$$

where l^{±}(k+2, qₖ) represent the positive and negative cable lengths for joint (k+2) as functions of a k^{th} joint angle, qₖ.

A motor differential angle, Δθᵣ, to counteract the coupling effect may be determined as

$${Δθ}_{r} = \frac{Δ {l}_{coupling} \cdot 2 π}{{p}_{n}}$$

where pₙ is representative of a cable pitch, p, for an n^{th} joint. To obtain a compensating motor command angle, Δθ_{comp}, the motor differential angle, Δθᵣ, may be split equally between the antagonistic motor pair:

$${Δθ}_{comp} = \frac{{Δθ}_{r}}{2} .$$

In routine operation, the processor of the proximal driving unit 502 may determine one or more desired motor command angles, e.g., θₙ⁺ and θₙ⁻. To implement a real-time, feed-forward compensation strategy, the processor of the proximal driving unit 502 may add the compensating motor command angle, Δθ_{comp}, to the desired motor command angles, θₙ⁺, in a control loop:

$${θ}_{n}^{\pm } = {θ}_{n}^{\pm } \pm {Δθ}_{comp} .$$

This feed-forward compensation strategy may be shown to establish that distal joints remain stationary during proximal joint motions, thereby enabling decoupled and intuitive control of the tool 202.

For the tensile coupling, the path length change may be determined. In some instances, compensation for tensile couplings may be neglected.

To create a model for the kinematic coupling, an empirical model may be created for each coupling pair. A dataset of the angular position of an independent joint and a dependent joint may be recorded across the range of motion of the independent joint. From this dataset, a fourth-degree polynomial model may be fit to describe a relationship between the angle of the dependent joint and the angle of the independent joint. The effective radius of the downstream link may be used to derive a function, ƒ(Q), representative of a change in path length of the dependent joint as a function of the independent joint, Q.

Although a combination of features is shown in the illustrated embodiments, not all of them need to be combined to realize the benefits of various embodiments of this disclosure. In other words, a system or method designed according to an embodiment of this disclosure will not necessarily include all of the features shown in any one of the Figures or all of the portions schematically shown in the Figures. Moreover, selected features of one example embodiment may be combined with selected features of other example embodiments.

Although this disclosure has been described with reference to illustrative embodiments, this description is not intended to be construed in a limiting sense. Various modifications and combinations of the illustrative embodiments, as well as other embodiments of the disclosure, will be apparent to persons skilled in the art upon reference to the description. It is therefore intended that the appended claims encompass any such modifications or embodiments.

In the present disclosure, the terms “a” and “an” are defined to mean “at least one.” That is, these terms do not exclude a plural number of items, unless stated otherwise.

In the present disclosure, terms such as “substantially,” “generally” and “about,” which modify a value, condition or characteristic of a feature of an example embodiment, should be understood to mean that the value, condition or characteristic is defined within tolerances that are acceptable for the proper operation of the example embodiment for its intended application.

In the present disclosure, unless stated otherwise, the terms “connected” and “coupled,” and derivatives and variants thereof, refer herein to any structural or functional connection or coupling, either direct or indirect, between two or more elements. For example, the connection or coupling between the elements can be acoustical, mechanical, optical, electrical, thermal, logical or any combinations thereof.

In the present disclosure, expressions such as “match,” “matching” and “matched,” including variants and derivatives thereof, are intended to refer herein to a condition in which two or more elements are either the same or within some predetermined tolerance of each other. That is, these terms are meant to encompass not only “exactly” or “identically” matching the two elements but also “substantially,” “approximately” or “subjectively” matching the two or more elements, as well as providing a higher or best match among a plurality of matching possibilities.

In the present disclosure, the expression “based on” is intended to mean “based at least partly on.” That is, this expression can mean “based solely on” or “based partially on” and, so, should not be interpreted in a limited manner. More particularly, the expression “based on” could also be understood as meaning “depending on,” “representative of,” “indicative of,” “associated with” or similar expressions.

In the present disclosure, the terms “system” and “network” may be used interchangeably in different embodiments of this application. “At least one” means one or more and “a plurality of” means two or more. The term “and/or” describes an association relationship of associated objects and indicates that three relationships may exist. For example, A and/or B may indicate the following three cases: only A exists; both A and B exist; and only B exists; where A and B may be singular or plural. The character “/” indicates an “or” relationship between associated objects. “At least one of the following items (pieces)” or a similar expression thereof indicates any combination of these items, including a single item (piece) or any combination of a plurality of items (pieces). For example, “at least one of A, B, or C” includes: only A; only B; only C; A and B; A and C; B and C; or A, B, and C. “at least one of A, B, and C” may also be understood as including: only A; only B; only C; A and B; A and C; B and C; or A, B, and C. In addition, unless otherwise specified, ordinal numbers such as “first” and “second” in embodiments of this application are used to distinguish between a plurality of objects and are not used to limit a sequence, a time sequence, priorities, or importance of the plurality of objects.

A person skilled in the art should understand that embodiments of this application may be provided as a method, an apparatus (or system), computer-readable storage medium, or a computer program product. Therefore, this application may use a form of a hardware-only embodiment, a software-only embodiment, or an embodiment with a combination of software and hardware. Moreover, this application may use a form of a computer program product that is implemented on one or more computer-usable storage media (including but not limited to a disk memory, an optical memory, and the like) that include computer-usable program code.

It is clear that a person skilled in the art can make various modifications and variations to this application without departing from the scope of this disclosure. This application is intended to cover these modifications and variations of this disclosure provided that they fall within the scope of protection defined by the following claims and their equivalent technologies.

1. A robotic tool comprising:
a driving unit including a plurality of tool actuators;
a tool shaft connected to the driving unit;
a three-degree-of-freedom (three-DOF) elbow joint arranged to connect the tool shaft to a secondary shaft, the three-DOF elbow joint configured for control by at least one tool actuator among the plurality of tool actuators; and
a three-DOF wrist joint connected to the secondary shaft, the three-DOF wrist joint configured for control by at least one tool actuator among the plurality of tool actuators.

2. The robotic tool of claim 1, wherein the degrees-of-freedom for the three-DOF wrist joint include wrist pitch, wrist yaw and wrist grip.

3. The robotic tool of claim 2, wherein the wrist joint has a range of motion of ±90 degrees in at least one of the degrees-of-freedom.

4. The robotic tool of claim 1, wherein the degrees-of-freedom for the three-DOF elbow joint include elbow pitch, elbow yaw and elbow roll.

5. The robotic tool of claim 4, wherein the elbow joint has a range of motion of ±90 degrees for pitch and yaw and ±180 degrees for roll in at least one of the degrees-of-freedom.

6. The robotic tool of claim 1, wherein the tool actuator comprises:
a lead screw;
a nut mounted on the lead screw;
a slider attached to the nut; and
a cable attached to the slider;
wherein rotation of the lead screw causes the nut to move linearly, thereby pulling or releasing the cable.

7. The robotic tool of claim 6, further comprising a guiding rail constraining rotation of the slider about a longitudinal axis of the lead screw.

8. The robotic tool of claim 6, wherein at least one tool actuator among the plurality of tool actuators comprises an actuation module configured to actuate the lead screw.

9. The robotic tool of claim 6, wherein the cable is, at least in part, made of tungsten.

10. The robotic tool of claim 6, wherein the cable is, at least in part, made of stainless steel, nylon or any metal or plastic or polymer.

11. The robotic tool of claim 6, wherein the elbow joint has a sandwich link architecture wherein proximal links of the elbow joint are split.

12. The robotic tool of claim 11, wherein each link among the links defines a guide channel for the cable.

13. The robotic tool of claim 11, wherein each link among the links defines a central bushing.

14. The robotic tool of claim 1, further comprising a decoupling mechanism for decoupling the driving unit to a robotic arm.

15. The robotic tool of claim 14, wherein the driving unit is configured to respond to actuation by the robotic arm.

16. The robotic tool of claim 1, wherein the driving unit is adapted to tension the cable.

17. The robotic tool of claim 1, wherein the driving unit is adapted to implement a strategy for compensating for coupling between the wrist joint and the elbow joint.

18. The robotic tool of claim 17, wherein the compensating comprises:
determining a desired motor command angle;
determining a measure of coupling effect between the wrist joint and the elbow joint;
determining, based on the measure of the coupling effect, a compensating motor command angle; and
applying, to the desired motor command angle, compensation based on the compensating motor command angle.

19. The robotic tool of claim 18, wherein the measure of the coupling effect comprises a induced length difference in the cable.

20. The robotic tool of claim 18, wherein the determining the compensating motor command angle comprises determining a motor differential angle and a pitch of the cable.
